SEQUENCE LISTING (29 sequences)

SEQ ID NO: 1 - protein, 663 aa, Artificial
  synthetic polypeptide
MGSSKSKPKDGGGGSSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTGSGGSGGMEPVDPRLEPWKHPGSQPKTACTNCYCKKCCFHCQVCFITKALGISYGRKKRRQRRRPPQGSQTHQVSLSKQPGSIATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 2 - DNA, 2013 bp, Artificial
  synthetic oligonucleotide
aagcttgccaccatgggcagcagtaagagtaaaccgaaagatggaggcggtgggtcatctatcacctcagctgttttgcagtctggttttaggaaaatggcgttccccagcggtaaagttgaaggatgtatggtccaagtaacctgtggtaccactacccttaatgggctttggttggacgacgtagtctactgcccccgacacgtaatctgcactagtgaggatatgcttaatcccaattacgaagaccttttgattcggaaatccaatcacaacttcctggtccaagcgggcaacgtccaactcagggttattggacatagtatgcagaattgcgtactgaagctcaaagtcgatactgcaaaccccaagacgcccaagtataaattcgtccgaatccaaccaggccaaacattttccgtattggcttgctataatggaagccccagcggtgtctaccaatgtgcaatgagaccaaactttacgataaagggttcatttctgaacggctcttgcggttccgttggttttaacatcgactatgactgtgtatccttttgctacatgcaccatatggaactccctaccggtgtccacgccggtacagatctggaaggaaatttctacggtccgttcgttgaccggcaaaccgcgcaagcggctggaaccgacacaacgattacagtgaatgtgctcgcgtggctgtacgcagcagtcataaacggagacaggtggtttctgaaccgatttacgacgactctcaatgacttcaaccttgttgcgatgaagtacaattacgagccactcacccaggaccatgttgatatcctgggtcccctcagtgcccagacagggatcgcagttctcgatatgtgcgcgtcactgaaggagcttctccaaaatggaatgaatgggcggaccatacttggttccgcactcctcgaagatgaatttactccatttgacgtggtcagacaatgcagtggggtcactttccagagtgcagtgaaaagaacaatcaagggtaccgggtcagggggcagcggagggatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcagccgggatccatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgc

SEQ ID NO: 3 - DNA, 57 bp, Artificial
  synthetic oligonucleotide
gtgggtcatctatcacctcagctgttttgcagtctggttttaggaaaatggcgttcc

SEQ ID NO: 4 - DNA, 60 bp, Artificial
  synthetic oligonucleotide
ccccctgacccggtacccttgattgttcttttcactgcactctggaaagtgaccccactg

SEQ ID NO: 5 - DNA, 57 bp, Artificial
  synthetic oligonucleotide
gtgggtcatctatcacctcagctgttttggcttctggttttaggaaaatggcgttcc

SEQ ID NO: 6 - DNA, 60 bp, Artificial
  synthetic oligonucleotide
ccccctgacccggtacccttgattgttcttttcactgcactcgcgaaagtgaccccactg

SEQ ID NO: 7 - DNA, 60 bp, Artificial
  synthetic oligonucleotide
agaacaatcaagggtaccgggtcagggggcagcggagggatggagccagtagatcctaga

SEQ ID NO: 8 - DNA, 47 bp, Artificial
  synthetic oligonucleotide
ggtggcgatggatcccggctgctttgatagagaaacttgatgagtct

SEQ ID NO: 9 - DNA, 47 bp, Artificial
  synthetic oligonucleotide
agactcatcaagtttctctatcaaagcagccgggatccatcgccacc

SEQ ID NO: 10 - DNA, 37 bp, Artificial
  synthetic oligonucleotide
gactcgagcggccgctttacttgtacagctcgtccat

SEQ ID NO: 11 - DNA, 72 bp, Artificial
  synthetic oligonucleotide
aagcttgccaccatgggcagcagtaagagtaaaccgaaagatggaggcggtgggtcatctatcacctcagct

SEQ ID NO: 12 - protein, 8 aa, Artificial
  synthetic polypeptide
AVLQSGFR

SEQ ID NO: 13 - protein, 8 aa, Artificial
  synthetic polypeptide
VTFQSAVK

SEQ ID NO: 14 - protein, 8 aa, Artificial
  synthetic polypeptide
ATVQSKMS

SEQ ID NO: 15 - protein, 8 aa, Artificial
  synthetic polypeptide
ATLQAIAS

SEQ ID NO: 16 - protein, 8 aa, Artificial
  synthetic polypeptide
VKLQNNEL

SEQ ID NO: 17 - protein, 8 aa, Artificial
  synthetic polypeptide
VRLQAGNA

SEQ ID NO: 18 - protein, 8 aa, Artificial
  synthetic polypeptide
PMLQSADA

SEQ ID NO: 19 - protein, 8 aa, Artificial
  synthetic polypeptide
TVLQAVGA

SEQ ID NO: 20 - protein, 8 aa, Artificial
  synthetic polypeptide
ATLQAENV

SEQ ID NO: 21 - protein, 8 aa, Artificial
  synthetic polypeptide
TRLQSLEN

SEQ ID NO: 22 - protein, 8 aa, Artificial
  synthetic polypeptide
PKLQSSQA

SEQ ID NO: 23 - protein, 973 aa, Artificial
  synthetic polypeptide
MGSSKSKPKDGGGGSSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTGSGGSGGMEPVDPRLEPWKHPGSQPKTACTNCYCKKCCFHCQVCFITKALGISYGRKKRRQRRRPPQGSQTHQVSLSKQPGSIATEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 24 - DNA, 2943 bp, Artificial
  synthetic oligonucleotide
aagcttgccaccatgggcagcagtaagagtaaaccgaaagatggaggcggtgggtcatctatcacctcagctgttttgcagtctggttttaggaaaatggcgttccccagcggtaaagttgaaggatgtatggtccaagtaacctgtggtaccactacccttaatgggctttggttggacgacgtagtctactgcccccgacacgtaatctgcactagtgaggatatgcttaatcccaattacgaagaccttttgattcggaaatccaatcacaacttcctggtccaagcgggcaacgtccaactcagggttattggacatagtatgcagaattgcgtactgaagctcaaagtcgatactgcaaaccccaagacgcccaagtataaattcgtccgaatccaaccaggccaaacattttccgtattggcttgctataatggaagccccagcggtgtctaccaatgtgcaatgagaccaaactttacgataaagggttcatttctgaacggctcttgcggttccgttggttttaacatcgactatgactgtgtatccttttgctacatgcaccatatggaactccctaccggtgtccacgccggtacagatctggaaggaaatttctacggtccgttcgttgaccggcaaaccgcgcaagcggctggaaccgacacaacgattacagtgaatgtgctcgcgtggctgtacgcagcagtcataaacggagacaggtggtttctgaaccgatttacgacgactctcaatgacttcaaccttgttgcgatgaagtacaattacgagccactcacccaggaccatgttgatatcctgggtcccctcagtgcccagacagggatcgcagttctcgatatgtgcgcgtcactgaaggagcttctccaaaatggaatgaatgggcggaccatacttggttccgcactcctcgaagatgaatttactccatttgacgtggtcagacaatgcagtggggtcactttccagagtgcagtgaaaagaacaatcaagggtaccgggtcagggggcagcggagggatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcagccgggatccatcgccaccgaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaagcggccgc

SEQ ID NO: 25 - protein, 969 aa, Artificial
  synthetic polypeptide
MGSSKSKPKDGGGGSVSYGSTLQAGLRKMAQPSGFVEKCVVRVCYGNTVLNGLWLGDIVYCPRHVIASNTTSAIDYDHEYSIMRLHNFSIISGTAFLGVVGATMHGVTLKIKVSQTNMHTPRHSFRTLKSGEGFNILACYDGCAQGVFGVNMRTNWTIRGSFINGACGSPGYNLKNGEVEFVYMHQIELGSGSHVGSSFDGVMYGGFEDQPNLQVESANQMLTVNVVAFLYAAILNGCTWWLKGEKLFVEHYNEWAQANGFTAMNGEDAFSILAAKTGVCVERLLHAIQVLNNGFGGKQILGYSSLNDEFSINEVVKQMFGVNLQSGKTTSMFGTGSGGSGGMEPVDPRLEPWKHPGSQPKTACTNCYCKKCCFHCQVCFITKALGISYGRKKRRQRRRPPQGSQTHQVSLSKQPGSIATEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 26 - DNA, 2931 bp, Artificial
  synthetic oligonucleotide
aagcttgccaccatgggcagcagtaagagtaaaccgaaagatggaggcggtgggtcagtatcttatggctcaacgctccaagccggcttgcgcaagatggcccaacccagtggctttgttgagaaatgtgtcgtgcgcgtgtgctatggcaatactgtcctcaacggtctctggctcggcgatatcgtgtattgcccgcggcatgtaatagcatctaatacaacttcagccattgattacgaccatgagtattcaataatgagacttcataatttcagtattataagcgggaccgccttcctgggcgtcgttggggctacgatgcatggggttactctgaagataaaggtttcccaaacaaatatgcacacgccccgccattcttttcggaccctgaagtctggcgagggatttaacattctggcgtgttacgacggatgcgcacaaggagttttcggagtaaacatgagaacaaattggactattcggggctcttttatcaacggcgcatgtggtagtcctggctacaatcttaaaaatggggaagtagagtttgtttatatgcaccagatcgagctggggtcaggttcccacgttggaagctcttttgatggggttatgtatggtggctttgaggaccaacccaatttgcaggtggaaagtgcgaaccaaatgttgacggtcaatgtcgtagctttcctctatgccgctatacttaacggctgtacttggtggttgaaaggcgagaaactgttcgtggaacattacaacgaatgggcgcaagccaacggattcactgccatgaacggcgaggatgctttctcaatactggcggccaaaaccggggtgtgtgtggaaagactccttcacgcaattcaggtactgaacaatggcttcggtgggaaacaaatcttgggatatagcagccttaatgatgaattctcaatcaacgaggtcgtcaaacaaatgtttggagtgaacctccaatctggtaagactacatccatgtttggtaccgggtcagggggcagcggagggatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcagccgggatccatcgccaccgaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaagcggccgc

SEQ ID NO: 27 - protein, 970 aa, Artificial
  synthetic polypeptide
MGSSKSKPKDGGGGSISYNSTLQSGLKKMAQPSGCVERCVVRVCYGSTVLNGVWLGDTVTCPRHVIAPSTTVLIDYDHAYSTMRLHNFSVSHNGVFLGVVGVTMHGSVLRIKVSQSNVHTPKHVFKTLKPGDSFNILACYEGIASGVFGVNLRTNFTIKGSFINGACGSPGYNVRNDGTVEFCYLHQIELGSGAHVGSDFTGSVYGNFDDQPSLQVESANLMLSDNVVAFLYAALLNGCRWWLCSTRVNVDGFNEWAMANGYTSVSSVECYSILAAKTGVSVEQLLASIQHLHEGFGGKNILGYSSLCDEFTLAEVVKQMYGVNLQSGKVIFGLGTGSGGSGGMEPVDPRLEPWKHPGSQPKTACTNCYCKKCCFHCQVCFITKALGISYGRKKRRQRRRPPQGSQTHQVSLSKQPGSIATEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 28 - DNA, 2931 bp, Artificial; identical to SEQ ID NO: 26
  synthetic oligonucleotide

SEQ ID NO: 29 - protein, 4 aa, Artificial
  synthetic polypeptide
AVLQ

(19) United States
(12) Patent Application Publication
(10) Pub. No.: US 2024/0011107 A1
(43) Pub. Date: Jan. 11, 2024

(54) LIVE CELL ASSAY FOR PROTEASE INHIBITION

(71) Applicant: Regents of the University of Minnesota, Minneapolis, MN (US)

(72) Inventors: Reuben S. Harris, Helotes, TX (US); Seyed Arad Moghadasi, Minneapolis, MN (US); Jordan Becker, Saint Paul, MN (US)

(21) Appl. No.: 18/035,021

(22) PCT Filed: Nov. 12, 2021
(86) PCT No.: PCT/US2021/057723

§ 371 Date: May 2, 2023

Related U.S. Application Data
(60) Provisional application No. 63/108,611, filed on Nov. 2, 2020.

(51) Int. Cl. C12Q 1/6897; C12Q 1/37

(52) U.S. Cl. CPC C12Q 1/6897; C12Q 1/37; G01N 2333/8107; G01N 2333/9513

(57) ABSTRACT

Materials and methods for identifying inhibitors of protease activity are provided herein. For example, this document provides materials and methods that can be used to identify inhibitors of a protease (e.g., SARS-CoV-2 M^{pro}).

### CROSS-REFERENCE TO RELATED APPLICATIONS

This application claims benefit of priority from U.S. Provisional Application Ser. No. 63/108,611, filed on Nov. 2, 2020.

### STATEMENT AS TO FEDERALLY SPONSORED RESEARCH

This invention was made with government support under CA234228 and AI064046 awarded by the National Institutes of Health. The government has certain rights in the invention.

### TECHNICAL FIELD

This document relates to materials and methods for identifying inhibitors of protease activity. For example, this document provides materials and methods that can be used to identify inhibitors of proteases such as SARS-CoV-2 M^{pro}.

### BACKGROUND

The main protease (M^{pro}) of SARS-CoV-2 is required to cleave the viral polyprotein into precise functional units for virus replication and pathogenesis. Viral proteases can effectively serve as targets for antiviral therapies (Hazuda et al., Ann NY Acad Sci 1291:69-76, 2013; Luna et al., Curr Opin Virol 35:27-34, 2019; and Yilmaz et al., Trends Microbiol 24:547-557, 2016). SARS-CoV-2 has two proteases—a Papain-Like protease (PL^{Pro}, Nsp3) and a Main protease/3C-Like protease (M^{pro}, 3CL^{pro}, Nsp5), which are responsible for three and eleven viral polyprotein cleavage events, respectively (Fehr and Perlman, Methods Mol Biol 1282:1-23, 2015; Hilgenfeld, FEBS J 281:4085-4096, 2014; Fung and Liu, Annu Rev Microbiol 73:529-557, 2019; and Wang et al., Methods Mol Biol 2203:1-29, 2020). These cleavage events are essential for virus replication and pathogenesis, and the proteases therefore have been under investigation for the development of drugs to combat the COVID-19 pandemic. Many biochemical assays are available for measuring SARS-CoV-2 protease activity (see, e.g., Fu et al., Nat Commun 11:4417, 2020; Vuong et al., Nat Commun 11:4282, 2020; and Jin et al., Nature 582:289-293, 2020), but specific and sensitive cellular assays are lacking.

### SUMMARY

This document is based, at least in part, on the development of a quantitative, gain-of-function reporter for MP^{pro} function in living cells, and on the development of methods for using the reporter to indicate levels of protease inhibition (e.g., by genetic or chemical means) as exhibited by, for example, strong enhanced green fluorescent protein (eGFP) fluorescence. The methods and materials disclosed herein provide a robust gain-of-function system that can be used to readily distinguish between inhibitor potencies, and can be scaled-up to high-throughput platforms for drug testing.

In a first aspect, this document features a nucleic acid construct encoding a modular reporter polypeptide, wherein the modular reporter polypeptide comprises, consists of, or consists essentially of, in order from N-terminus to C-terminus: an optional myristoylation motif, a protease polypeptide, an optional transactivator of transcription (Tat) sequence, and a reporter polypeptide. The myristoylation motif can be a Src myristoylation motif, an ADP-ribosylation factor (ARF) GTPase myristoylation motif, a human immunodeficiency virus-1 (HIV-1) Gag myristoylation motif, or a myristoylated alanine-rich C kinase substrate (MARCKS) myristoylation motif. The protease can be a viral protease. The protease polypeptide can be a SARS-CoV-2 M^{pro} polypeptide, a MERS M^{pro} polypeptide, a SARS M^{pro} polypeptide, a hepatitis C virus (HCV) NS3/4a protease polypeptide, a picornavirus 3C protease polypeptide, a HCoV-229E M^{pro} polypeptide, or a HCoV-NL63 M^{pro} polypeptide. The protease can be SARS-CoV-2 M^{pro}. The Tat sequence can include amino acids 1 to 72 of HIV-1 Tat. The reporter can be a fluorescent polypeptide. The fluorescent polypeptide can be a green fluorescent polypeptide (GFP), a red fluorescent polypeptide (RFP), or a yellow fluorescent polypeptide (YFP). The fluorescent polypeptide can be an enhanced GFP polypeptide (eGFP). The reporter can be a luminescent polypeptide (e.g., luciferase). The modular reporter polypeptide can further include a first linker sequence between the myristoylation motif and the protease polypeptide, a second linker sequence between the protease polypeptide and the Tat sequence, and a third linker sequence between the Tat sequence and the reporter polypeptide. The myristoylation motif can include the amino acid sequence set forth in residues 1 to 10 of SEQ ID NO:1, or an amino acid sequence that is at least 90% identical to the sequence set forth in residues 1 to 10 of SEQ ID NO:1. The protease polypeptide can include the amino acid sequence set forth in residues 16 to 337 of SEQ ID NO:1, residues 16 to 333 of SEQ ID NO:25, or residues 16 to 334 of SEQ ID NO:27, or an amino acid sequence that is at least 90% identical to the sequence set forth in residues 16 to 337 of SEQ ID NO:1 residues 16 to 333 of SEQ ID NO:25, or residues 16 to 334 of SEQ ID NO:27. The Tat sequence can include the amino acid sequence set forth in residues 347 to 418 of SEQ ID NO:1, or an amino acid sequence that is at least 90% identical to the sequence set forth in residues 347 to 418 of SEQ ID NO:1. The reporter polypeptide can include the amino acid sequence set forth in residues 425 to 663 of SEQ ID NO:1 or residues 425 to 973 of SEQ ID NO:23, or an amino acid sequence that is at least 90% identical to the sequence set forth in residues 425 to 663 of SEQ ID NO:1 or residues 425 to 973 of SEQ ID NO:23.

In another aspect, this document features a method for identifying an agent as being a protease inhibitor. The method can include: providing a cell transfected with and expressing a nucleic acid construct encoding a modular reporter polypeptide, where the modular reporter polypeptide comprises, consists essentially of, or consists of, in order from N-terminus to C-terminus: an optional myristoylation motif, a protease polypeptide, an optional Tat sequence, and a reporter polypeptide; contacting the cell with the agent; determining a level of reporter activity in the cell; comparing the level of reporter activity in the cell to a control level of reporter activity; and identifying the agent as being an inhibitor of the protease when the level of reporter activity in the cell is higher than the control level of reporter activity. The reporter activity can be fluorescence or luminescence. The control level of reporter activity can be a level of reporter activity in the cell determined prior to the contacting step. The control level of reporter activity can be a level of reporter activity in a corresponding cell transfected with and expressing the nucleic acid construct but not contacted with the agent. The myristoylation motif can be a Src myristoylation motif, an ARF GTPase myristoylation motif, a HIV-1 Gag myristoylation motif, or a MARCKS myristoylation motif. The protease can be a viral protease. The protease polypeptide can be a SARS-CoV-2 M^{pro} polypeptide, a MERS M^{pro} polypeptide, a SARS MP″ polypeptide, a HCV NS3/4a protease polypeptide, a picornavirus 3C protease polypeptide, a HCoV-229E M^{pro} polypeptide, or a HCoV-NL63 M^{pro} polypeptide. The protease can be SARS-CoV-2 M^{pro}. The Tat sequence can include amino acids 1 to 72 of HIV-1 Tat. The reporter can be a fluorescent polypeptide. The fluorescent polypeptide can be a GFP, a RFP, or a YFP. The fluorescent polypeptide can be an eGFP. The reporter polypeptide can be a luminescent polypeptide (e.g., luciferase). The modular reporter polypeptide can further include a first linker sequence between the myristoylation motif and the protease polypeptide, a second linker sequence between the protease polypeptide and the Tat sequence, and a third linker sequence between the Tat sequence and the fluorescent reporter polypeptide. The myristoylation motif can include the amino acid sequence set forth in residues 1 to 10 of SEQ ID NO:1, or an amino acid sequence that is at least 90% identical to the sequence set forth in residues 1 to 10 of SEQ ID NO:1. The protease polypeptide can include the amino acid sequence set forth in residues 16 to 337 of SEQ ID NO:1, residues 16 to 333 of SEQ ID NO:25, or residues 16 to 334 of SEQ ID NO:27, or an amino acid sequence that is at least 90% identical to the sequence set forth in residues 16 to 337 of SEQ ID NO:1, residues 16 to 333 of SEQ ID NO:25, or residues 16 to 334 of SEQ ID NO:27. The Tat sequence can include the amino acid sequence set forth in residues 347 to 418 of SEQ ID NO:1, or an amino acid sequence that is at least 90% identical to the sequence set forth in residues 347 to 418 of SEQ ID NO:1. The reporter polypeptide can include the amino acid sequence set forth in residues 425 to 663 of SEQ ID NO:1 or residues 425 to 973 of SEQ ID NO:23, or an amino acid sequence that is at least 90% identical to the sequence set forth in residues 425 to 663 of SEQ ID NO:1 or residues 425 to 973 of SEQ ID NO:23. The agent can be a small molecule or an anti-M^{pro} antibody.

In another aspect, this document features a method for identifying a protease as having a mutation that reduces activity of the protease. The method can include: providing a cell transfected with and expressing a nucleic acid construct encoding a modular reporter polypeptide, where the modular reporter polypeptide comprises, consists essentially of, or consists of, in order from N-terminus to C-terminus: an optional myristoylation motif, a protease polypeptide, where the amino acid sequence of the protease polypeptide includes a mutation with respect to a corresponding wild type protease polypeptide amino acid sequence, an optional Tat sequence, and a reporter polypeptide; determining a level of reporter activity in the cell; comparing the level of reporter activity in the cell to a control level of reporter activity; and identifying the agent as being an inhibitor of the protease when the level of reporter activity in the cell is higher than the control level of reporter activity. The reporter activity can be fluorescence or luminescence. The control level of reporter activity can be a level of reporter activity in a corresponding cell transfected with and expressing a nucleic acid construct that encodes a modular reporter polypeptide comprising a protease polypeptide with a wild type amino acid sequence. The myristoylation motif can be a Src myristoylation motif, an ARF GTPase myristoylation motif, a HIV-1 Gag myristoylation motif, or a MARCKS myristoylation motif. The protease can be a viral protease. The protease polypeptide can be a SARS-CoV-2 M^{pro} polypeptide, a MERS M^{pro} polypeptide, a SARS M^{pro} polypeptide, a HCV NS3/4a protease polypeptide, a picornavirus 3C protease polypeptide, a HCoV-229E M^{pro} polypeptide, or a HCoV-NL63 M^{pro} polypeptide. The protease can be SARS-CoV-2 M^{pro}. The Tat sequence can include amino acids 1 to 72 of HIV-1 Tat. The reporter can be a fluorescent polypeptide. The fluorescent polypeptide can be a GFP, a RFP, or a YFP. The fluorescent polypeptide can be an eGFP. The reporter can be a luminescent polypeptide (e.g., luciferase). The modular reporter polypeptide can further include a first linker sequence between the myristoylation motif and the protease polypeptide, a second linker sequence between the protease polypeptide and the Tat sequence, and a third linker sequence between the Tat sequence and the fluorescent reporter polypeptide. The myristoylation motif can include the amino acid sequence set forth in residues 1 to 10 of SEQ ID NO:1, or an amino acid sequence that is at least 90% identical to the sequence set forth in residues 1 to 10 of SEQ ID NO:1. The protease polypeptide can include the amino acid sequence set forth in residues 16 to 337 of SEQ ID NO:1, residues 16 to 333 of SEQ ID NO:25, or residues 16 to 334 of SEQ ID NO:27, or an amino acid sequence that is at least 90% identical to the sequence set forth in residues 16 to 337 of SEQ ID NO:1, residues 16 to 333 of SEQ ID NO:25, or residues 16 to 334 of SEQ ID NO:27. The Tat sequence can include the amino acid sequence set forth in residues 347 to 418 of SEQ ID NO:1, or an amino acid sequence that is at least 90% identical to the sequence set forth in residues 347 to 418 of SEQ ID NO:1. The reporter polypeptide can include the amino acid sequence set forth in residues 425 to 663 of SEQ ID NO:1 or residues 425 to 973 of SEQ ID NO:23, or an amino acid sequence that is at least 90% identical to the sequence set forth in residues 425 to 663 of SEQ ID NO:1 or residues 425 to 973 of SEQ ID NO:23.

In still another aspect, this document features a kit containing a nucleic acid construct that encodes a modular reporter polypeptide, where the modular reporter polypeptide comprises, consists essentially of, or consists of, in order from N-terminus to C-terminus: an optional myristoylation motif, a protease polypeptide, an optional Tat sequence, and a reporter polypeptide.

This document also features a kit containing a cell that contains a nucleic acid construct encoding a modular reporter polypeptide, where the modular reporter polypeptide comprises, consists essentially of, or consists of, in order from N-terminus to C-terminus: an optional myristoylation motif, a protease polypeptide, an optional HIV-1 Tat sequence, and a fluorescent reporter polypeptide. The kit nucleic acid construct can be stably integrated into the genome of the cell.

In the kits provided herein, the myristoylation motif can be a Src myristoylation motif, an ARF GTPase myristoylation motif, a HIV-1 Gag myristoylation motif, or a MARCKS myristoylation motif. The protease can be a viral protease. The protease polypeptide can be a SARS-CoV-2 M^{pro} polypeptide, a MERS M^{pro} polypeptide, a SARS M^{pro} polypeptide, a HCV NS3/4a protease polypeptide, a picornavirus 3C protease polypeptide, a HCoV-229E M^{pro} polypeptide, or a HCoV-NL63 M^{pro} polypeptide. The protease can be SARS-CoV-2 M^{pro}. The Tat sequence can include amino acids 1 to 72 of HIV-1 Tat. The reporter can be a fluorescent polypeptide. The fluorescent polypeptide can be a GFP, a RFP, or a YFP. The fluorescent polypeptide can be an eGFP. The reporter can be a luminescent polypeptide (e.g., luciferase). The modular reporter polypeptide can further include a first linker sequence between the myristoylation motif and the protease polypeptide, a second linker sequence between the protease polypeptide and the Tat sequence, and a third linker sequence between the Tat sequence and the fluorescent reporter polypeptide. The myristoylation motif can include the amino acid sequence set forth in residues 1 to 10 of SEQ ID NO:1, or an amino acid sequence that is at least 90% identical to the sequence set forth in residues 1 to 10 of SEQ ID NO:1. The protease polypeptide can include the amino acid sequence set forth in residues 16 to 337 of SEQ ID NO:1, residues 16 to 334 of SEQ ID NO:25, or residues 16 to 333 of SEQ ID NO:27, or an amino acid sequence that is at least 90% identical to the sequence set forth in residues 16 to 337 of SEQ ID NO:1, residues 16 to 334 of SEQ ID NO:25, or residues 16 to 333 of SEQ ID NO:27. The Tat sequence can include the amino acid sequence set forth in residues 347 to 418 of SEQ ID NO:1, or an amino acid sequence that is at least 90% identical to the sequence set forth in residues 347 to 418 of SEQ ID NO:1. The reporter polypeptide can include the amino acid sequence set forth in residues 425 to 663 of SEQ ID NO:1 or residues 425 to 973 of SEQ ID NO:23, or an amino acid sequence that is at least 90% identical to the sequence set forth in residues 425 to 663 of SEQ ID NO:1 or residues 425 to 973 of SEQ ID NO:23.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows the amino acid sequence for a Src-M^{pro}-Tat-eGFP polypeptide (SEQ ID NO:1) below a table indicating the location of particular domains within the polypeptide. Linker sequences are underlined. FIG. 1B shows the complete nucleotide sequence of the Src-M^{pro}-Tat-eGFP construct (SEQ ID NO:2), from the HindIII 5′ restriction site to the NotI 3′ restriction site. The sequence encodes the polypeptide domains detailed in the table in FIG. 1A. Untranslated sequences at the 5′ and 3′ ends are italicized, and sequences encoding the linkers are underlined. The DNA sequences for Src and M^{pro} are codon optimized for expression in human cells.

FIG. 2A shows the amino acid sequence for a Src-SARS2-M^{pro}-Tat-fLuc polypeptide (SEQ ID NO:23) below a table indicating the location of particular domains within the polypeptide. Linker sequences are underlined. FIG. 2B shows a nucleotide sequence for the Src-SARS2-M^{pro}-Tat-fLuc construct (SEQ ID NO:24). The sequence encodes the polypeptide domains detailed in the table in FIG. 2A. Untranslated sequences at the 5′ and 3′ ends are italicized, and sequences encoding the linkers are underlined. The DNA sequences for Src and M^{pro} are codon optimized for expression in human cells.

FIG. 3A shows the amino acid sequence for a Src-HCoV229E-M^{pro}-Tat-fLuc polypeptide (SEQ ID NO:25) below a table indicating the location of particular domains within the polypeptide. Linker sequences are underlined. FIG. 3B shows a nucleotide sequence for the Src-HCoV229E-M^{pro}-Tat-fLuc construct (SEQ ID NO:26). The sequence encodes the polypeptide domains detailed in the table in FIG. 3A. Untranslated sequences at the 5′ and 3′ ends are italicized, and sequences encoding the linkers are underlined. The DNA sequences for Src and M^{pro} are codon optimized for expression in human cells.

FIG. 4A shows the amino acid sequence for a Src-HCoV-NL63-M^{pro}-Tat-fLuc polypeptide (SEQ ID NO:27) below a table indicating the location of particular domains within the polypeptide. Linker sequences are underlined. FIG. 4B shows a nucleotide sequence for the Src-HCoV-NL63-M^{pro}-Tat-fLuc construct (SEQ ID NO:28). The sequence encodes the polypeptide domains detailed in the table in FIG. 4A. Untranslated sequences at the 5′ and 3′ ends are italicized, and sequences encoding the linkers are underlined. The DNA sequences for Src and M^{pro} are codon optimized for expression in human cells.

FIGS. 5A-5C show a gain-of-function system for SARS-CoV-2 M^{pro}inhibition in living cells. FIG. 5A is a schematic of the 4-part wild type (WT), catalytic mutant (C145A), and cleavage site mutant (CSM) chimeric constructs described herein (left), and a bar graph of the mean eGFP fluorescence intensity of the indicated constructs in 293T cells 48 hours post-transfection (right) [mean±SD of n=3 biologically independent experiments (individual data points shown); **, p<0.002 by unpaired student's t-test]. FIG. 5B is a series of representative fluorescent microscopy images of 293T cells expressing the indicated chimeric constructs (top). An NLS-mCherry plasmid was included in each reaction as a control for transfection and imaging (bottom). Scale bars are 100 μm. FIG. 5C shows an anti-eGFP immunoblot for the indicated Src-M^{pro}-Tat-eGFP constructs. A parallel anti-β-actin blot was used as a loading control.

FIGS. 6A-6E show that GC376 was more potent than boceprevir in blocking SARS-CoV-2 M^{pro}function in living cells. FIG. 6A is a histogram of the mean eGFP fluorescence intensity of the wild type M^{pro} chimeric construct in 293T cells incubated with 50 μM GC376, 50 μM boceprevir, or DMSO (mean±SD of n=3 biologically independent experiments; ***, p=0.0003, ****, p<0.0001 by unpaired student's t-test).

FIG. 6B is a graph plotting a dose response curve of GFP mean fluorescence intensity (MFI) in 293T cells transfected with WT Src-M^{pro}-Tat-eGFP and treated with the indicated concentrations of GC376. Quantification is mean±SD of the MFI from n=3 biologically independent experiments. FIG. 6C shows an anti-eGFP immunoblot indicating differential accumulation of Tat-eGFP and Src-M^{pro}-Tat-eGFP following incubation with the indicated amounts of GC376. A parallel anti-β-actin blot was done as a loading control. FIGS. 6D and 6E are representative fluorescent images of 293T cells expressing the wild type M^{pro} chimeric construct and treated with the indicated concentrations of GC376.

FIG. 7 is a series of representative fluorescent images of HeLa cells transfected with Src-M^{pro}-Tat-eGFP and treated with 50 μM GC376 or boceprevir (scale bars are 200 μm).

FIGS. 8A-8C illustrate a FlipGFP system for quantification of SARS-CoV-2 M^{pro} activity. FIG. 8A is a schematic showing a FlipGFP system (adapted from Zhang et al., J Am Chem Soc 141(11):4526-4530, 2019). Cleavage by SARS-CoV-2 M^{pro} (indicated by scissors) enables the split β strands 10 and 11 to flip from a parallel orientation into an antiparallel conformation, which reconstitutes GFP fluorescence. AVLQ sequence at the C-terminus of the antiparallel conformation, SEQ ID NO:29. FIG. 8B is a series of representative fluorescent images of 293T cells co-transfected with the C14 cleavage construct and either an M^{pro} or M^{pro}-C145A expression construct. mCherry was used as an internal control for visualization of transfected cells. FIG. 8C is a histogram plotting the fold change in mean GFP fluorescence intensity of 293T cells transfected with the indicated SARS-CoV-2 cleavage site constructs (C4-C14; SEQ ID NOS:12-22 respectively) and either an M^{pro} or M^{pro}-C145A expression construct (mean±SD of n=3 biologically independent experiments).

FIGS. 9A and 9B show reporter activity for a firefly luciferase-based assay system vs. an eGFP-based assay system. FIG. 9A is a graph plotting the signal fold change over background (DMSO) with the indicated concentrations of GC376 (n=3 with SEM indicated) for a luciferase-based reporter and an eGFP reporter. FIG. 9B is a graph plotting the signal fold change over background (DMSO) with the indicated concentrations of boceprevir (n=3 with SEM indicated) for a luciferase-based reporter and an eGFP reporter. The DMSO control (not shown) was normalized to 1.

FIGS. 10A and 10B show that diverse human coronavirus M^{pro} enzymes function in a luciferase-based reporter system and show differential inhibition by GC376 and boceprevir. FIG. 10A is a graph plotting the signal fold change over background (DMSO) at increasing concentrations of GC376 (n=3 with SEM indicated) for reporters containing SARS-CoV-2 HCoV-229E M^{pro}, and HCoV-NL63 M^{pro}. FIG. 10B is a graph plotting the signal fold change over background (DMSO) at increasing concentrations of boceprevir (n=3 with SEM indicated) for reporters containing SARS-CoV-2 M^{pro}, HCoV-229E M^{pro}, and HCoV-NL63 M^{pro}. The DMSO control (not shown) was normalized to 1.

### DETAILED DESCRIPTION

This document is based, at least in part, on the development of a robust, quantitative, gain-of-function reporter for protease function (or lack thereof) in living cells. The reporter provides a robust gain-of-function system that can be used to identify inhibitors and distinguish between inhibitor potencies, and can be scaled-up to high-throughput platforms for drug testing. In some cases, therefore, this document provides a modular reporter polypeptide. This document also provides nucleic acid constructs encoding the reporter, cells containing the nucleic acid constructs, and articles of manufacture containing the nucleic acid constructs and/or the cells. In addition, this document provides methods for using the nucleic acids and reporter polypeptides to indicate protease inhibition as exhibited by, for example, fluorescence of the reporter.

In some cases, this document provides fusion polypeptides that are modular reporters. The fusion polypeptides can include a protease polypeptide and a reporter polypeptide. In some cases, the fusion polypeptides also can include a myristoylation motif and/or a transactivator of transcription (Tat) sequence. In some cases, the fusion polypeptides can include, in order from N-terminus to C-terminus: protease-reporter, myristoylation motif-protease-reporter, protease-Tat sequence-reporter, or myristoylation motif-protease-Tat sequence-reporter. It is to be noted that in some cases, the fusion polypeptides can include a tag such as a FLAG® tag or a streptavidin tag in place of the reporter polypeptide.

The term “polypeptide” as used herein refers to a molecule of two or more subunit amino acids, regardless of post-translational modification (e.g., phosphorylation or glycosylation). The amino acid subunits may be linked by peptide bonds or other bonds such as, for example, ester or ether bonds. The term “amino acid” refers to either natural and/or unnatural or synthetic amino acids, including D/L optical isomers.

An “isolated” or “purified” polypeptide is a polypeptide that is separated to some extent from the cellular components with which it is normally found in nature (e.g., other polypeptides, lipids, carbohydrates, and nucleic acids). A purified polypeptide can yield a single major band on a non-reducing polyacrylamide gel. A purified polypeptide can be at least about 75% pure (e.g., at least about 80%, at least about 85%, at least about 90%, at least about 95%, at least about 97%, at least about 98%, at least about 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

When included, any appropriate myristoylation motif can be contained in the fusion polypeptides provided herein. In some cases, for example, a fusion polypeptide can be a Src myristoylation motif. Other suitable myristoylation motifs can be derived from, for example, ADP-ribosylation factor (ARF) GTPases, a human immunodeficiency virus (HIV) Gag polypeptide, and a myristoylated alanine-rich C kinase substrate (MARCKS) protein. See, e.g., Liu et al., Nature Struct Mol Biol 17:876-881, 2010; Reil et al., EMBO J 17(9):2699-2708, 1998; and Graff and Blackshear, Science 246(4929):503-506, 1989.

Any appropriate protease polypeptide can be included in the fusion polypeptides provided herein. In some cases, a fusion polypeptide can include a portion of a full-length protease protein, provided that the portion has protease activity in the absence of an inhibitor. In some cases, a fusion polypeptide can include an amino acid sequence from a viral protease. Non-limiting examples of protease polypeptides that can be included in a fusion polypeptide described herein include a SARS-Cov-2 M^{pro} polypeptide, a MERS M^{pro} polypeptide, a SARS M^{pro} polypeptide, a hepatitis C virus (HCV) NS3/4a protease, and a picornavirus 3C protease.

When included, any appropriate Tat sequence can be contained in the fusion polypeptides provided herein. For example, a fusion polypeptide can include a lentivirus (e.g., HIV-1) Tat amino acid sequence, or an amino acid sequence from another lentivirus (e.g., HIV-2 or SIV) Tat polypeptide. In some cases, the Tat portion of a fusion polypeptide provided herein can contain amino acids 1-72 of the HIV-1 Tat protein.

Any appropriate reporter polypeptide that provides a quantitative read-out can be optionally included in the fusion polypeptides provided herein. In some cases, for example, a reporter can be a fluorescent polypeptide or a luminescent polypeptide, or another polypeptide such as beta-galactosidase. Fluorescent polypeptides that can be used as reporters include in the fusion polypeptides provided herein include, without limitation, green fluorescent polypeptides (GFPs), such as enhanced GFP (eGFP), red fluorescent polypeptides (RFP), and yellow fluorescent polypeptides (YFP). Examples of luminescent polypeptides that can be used as reporters in the fusion polypeptides provided herein include, without limitation, luciferase and variants thereof (e.g., Firefly luciferase, Renilla luciferase, and NANOLUC® luciferase). Expression of reporter polypeptides in a cell can cause fluorescence or luminescence in the cell, which can be detected and quantitated using, for example, fluorescence microscopy, flow cytometry, or a luminometer.

In some cases, the fusion polypeptides provided herein can include a linker sequence between adjacent domains. For example, a fusion polypeptide can include a linker sequence between the myristoylation motif and the protease polypeptide, between the protease polypeptide and the Tat sequence, between the Tat sequence and the reporter, or any combination thereof. Any appropriate linker sequence can be used. In some cases, the linker(s) can be non-structured and flexible. When more than one linker is present in a fusion polypeptide, each linker can have a different sequence, or the linkers can have the same sequence. Suitable linker sequences can be, for example, from about 3 to about 20 amino acids in length (e.g., about 5 to about 18, about 7 to about 16, or about 10 to about 15 amino acids in length).

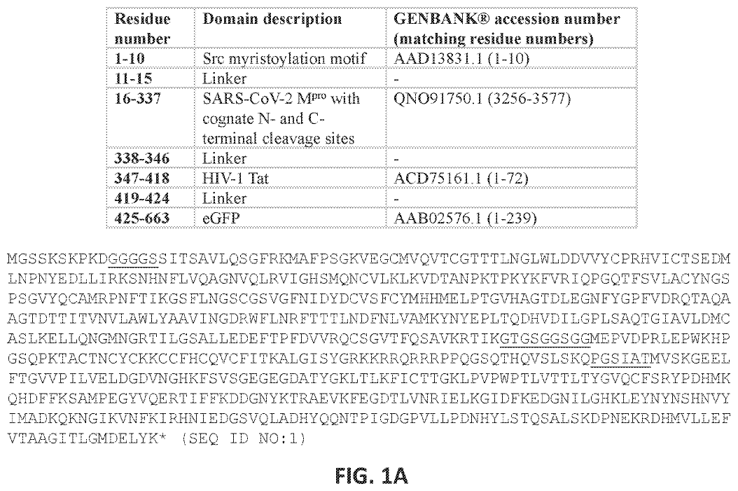

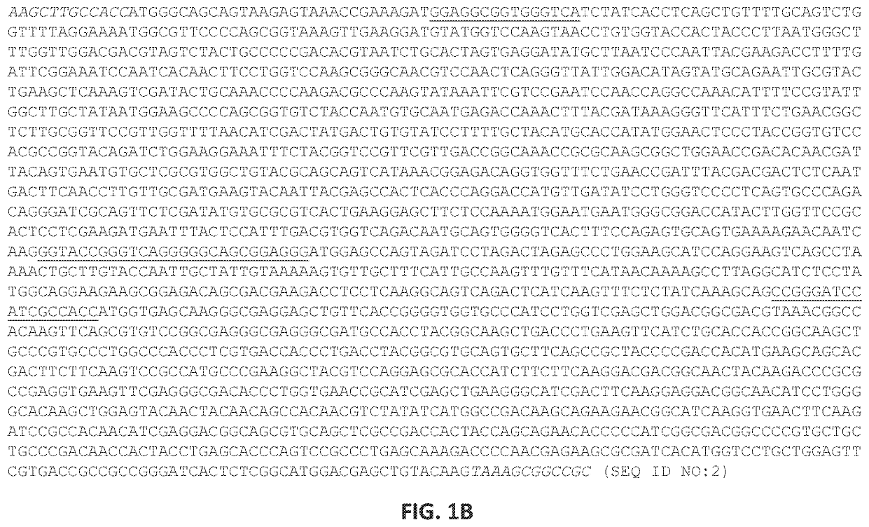

A representative amino acid sequence for an example of a fusion polypeptide provided herein is set forth in SEQ ID NO:1 (FIG. 1A); this representative polypeptide includes sequences from a Src myristoylation motif, SARS-CoV-2 M^{pro}, HIV-1 Tat, and eGFP. As indicated in the table in FIG. 1A, in some cases, a fusion polypeptide can include a myristoylation motif that includes amino acids 1 to 10 of SEQ ID NO:1, a protease polypeptide that includes amino acids 16 to 337 of SEQ ID NO:1, a HIV-1 Tat polypeptide that includes amino acids 347 to 418 of SEQ ID NO:1, and a fluorescent reporter (eGFP) polypeptide that includes amino acids 425 to 663 of SEQ ID NO:1. The fusion polypeptide sequence shown in FIG. 1A also includes linkers between adjacent domains (amino acids 11 to 15, 338 to 346, and 419 to 424 of SEQ ID NO:1). It is to be noted that the depicted linker sequences are non-limiting, and that other sequences can be used in place of those that are shown.

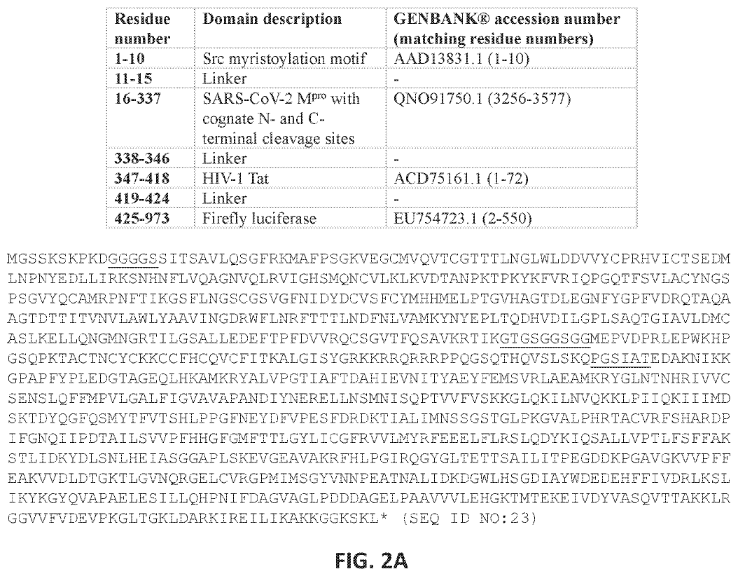

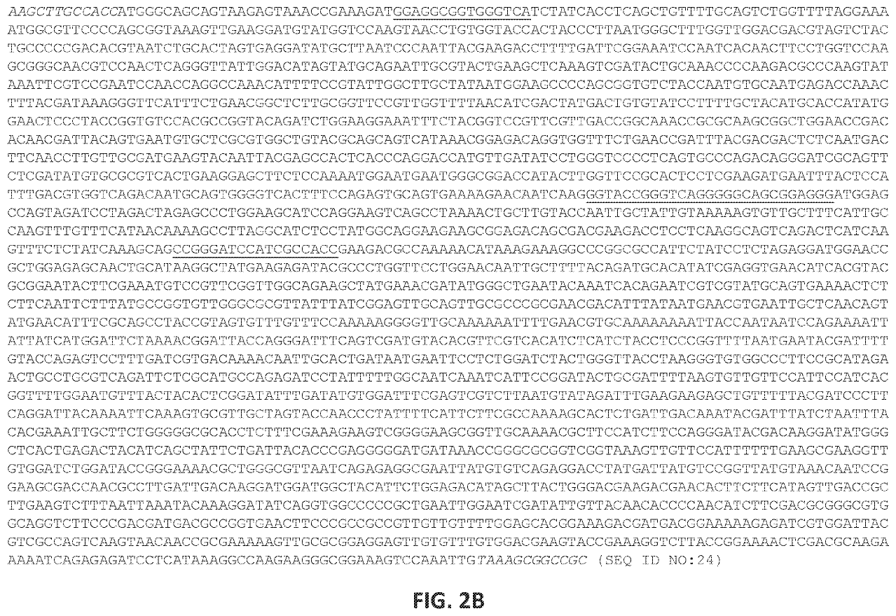

Another representative amino acid sequence for an example of a fusion polypeptide provided herein is set forth in SEQ ID NO:23 (FIG. 2A); this representative polypeptide includes sequences from a Src myristoylation motif, SARS-CoV-2 M^{pro}, HIV-1 Tat, and firefly luciferase. As indicated in the table in FIG. 2A, in some cases, a fusion polypeptide can include a myristoylation motif that includes amino acids 1 to 10 of SEQ ID NO:23, a protease polypeptide that includes amino acids 16 to 337 of SEQ ID NO:23, a HIV-1 Tat polypeptide that includes amino acids 347 to 418 of SEQ ID NO:23, and a luminescent reporter (luciferase) polypeptide that includes amino acids 425 to 973 of SEQ ID NO:23. The fusion polypeptide sequence shown in FIG. 2A also includes linkers between adjacent domains (amino acids 11 to 15, 338 to 346, and 419 to 424 of SEQ ID NO:23). It is to be noted that the depicted linker sequences are non-limiting, and that other sequences can be used in place of those that are shown.

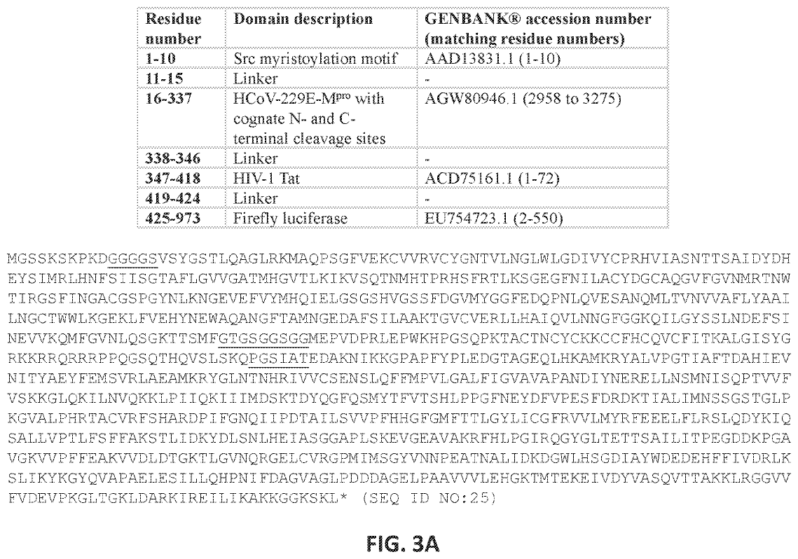

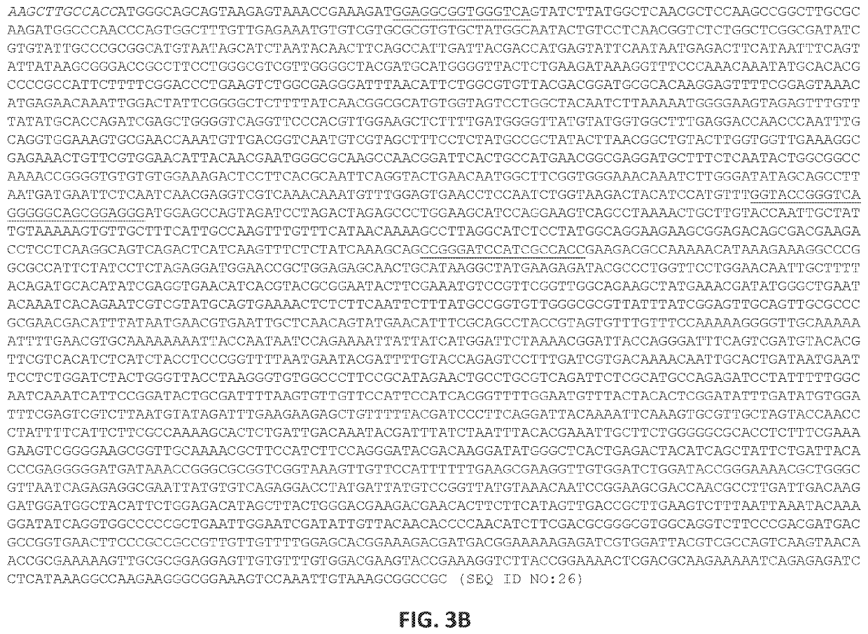

A further representative amino acid sequence for an example of a fusion polypeptide provided herein is set forth in SEQ ID NO:25 (FIG. 3A); this representative polypeptide includes sequences from a Src myristoylation motif, HCoV-229E M^{pro}, HIV-1 Tat, and luciferase. As indicated in the table in FIG. 3A, in some cases, a fusion polypeptide can include a myristoylation motif that includes amino acids 1 to 10 of SEQ ID NO:25, a protease polypeptide that includes amino acids 16 to 333 of SEQ ID NO:25, a HIV-1 Tat polypeptide that includes amino acids 343 to 414 of SEQ ID NO:25, and a luminescent reporter (luciferase) polypeptide that includes amino acids 421 to 969 of SEQ ID NO:25. The fusion polypeptide sequence shown in FIG. 3A also includes linkers between adjacent domains (amino acids 11 to 15, 334 to 342, and 415 to 420 of SEQ ID NO:25). It is to be noted that the depicted linker sequences are non-limiting, and that other sequences can be used in place of those that are shown.

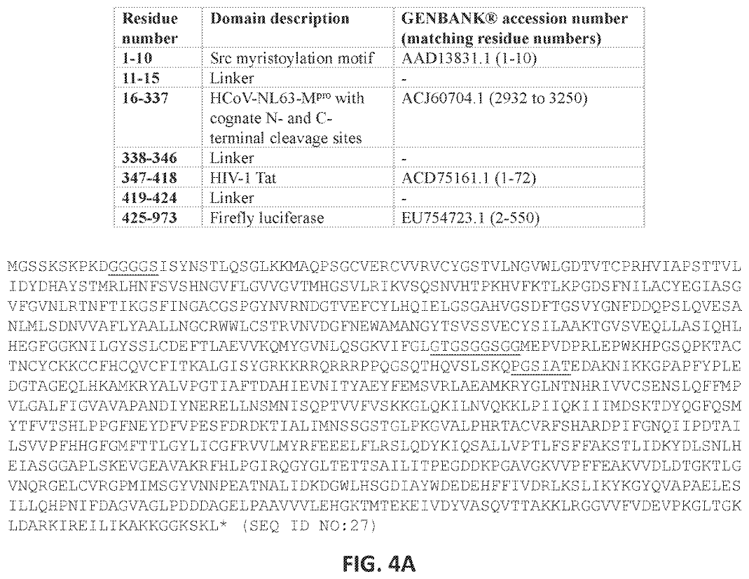

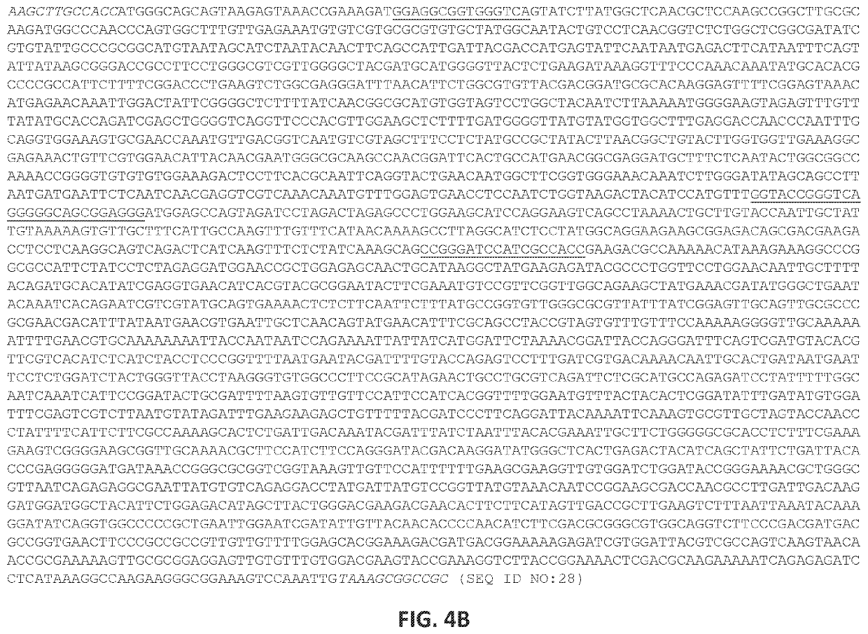

Another representative amino acid sequence for an example of a fusion polypeptide provided herein is set forth in SEQ ID NO:27 (FIG. 4A); this representative polypeptide includes sequences from a Src myristoylation motif, HCoV-NL63 M^{pro}, HIV-1 Tat, and eGFP. As indicated in the table in FIG. 4A, in some cases, a fusion polypeptide can include a myristoylation motif that includes amino acids 1 to 10 of SEQ ID NO:27, a protease polypeptide that includes amino acids 16 to 334 of SEQ ID NO:27, a HIV-1 Tat polypeptide that includes amino acids 344 to 415 of SEQ ID NO:27, and a luminescent reporter (luciferase) polypeptide that includes amino acids 422 to 970 of SEQ ID NO:27. The fusion polypeptide sequence shown in FIG. 4A also includes linkers between adjacent domains (amino acids 11 to 15, 335 to 343, and 416 to 421 of SEQ ID NO:27). It is to be noted that the depicted linker sequences are non-limiting, and that other sequences can be used in place of those that are shown.

In some cases, a fusion polypeptide can contain amino acid sequences that are variants (e.g., that contain one or more, two or more, three or more, four or more, or five or more substitutions, deletions, or additions) of the sequences set forth within SEQ ID NOS:1, 23, 25, and 27.

For example, a fusion polypeptide can include a myristoylation amino acid sequence that is at least 90% identical to the amino acid sequence set forth in residues 1 to 10 of SEQ ID NOS:1, 23, 25, and 27.

In some cases, a fusion polypeptide can include a SARS-CoV-2 M^{pro} amino acid sequence that is at least 90% (e.g., at least 91%, at least 92%, at least 93%, at least 94%, at least 95%, at least 96%, at least 97%, at least 98%, or at least 99%, but not 100%) identical to the sequence set forth in residues 16 to 337 of SEQ ID NO:1, with the proviso that the SARS-CoV-2 M^{pro} polypeptide has detectable activity in the absence of an inhibitor. In some cases, a fusion polypeptide can include a HCoV-229E M^{pro} amino acid sequence that is at least 90% (e.g., at least 91%, at least 92%, at least 93%, at least 94%, at least 95%, at least 96%, at least 97%, at least 98%, or at least 99%, but not 100%) identical to the sequence set forth in residues 16 to 333 of SEQ ID NO:25, with the proviso that the HCoV-229E M^{pro} polypeptide has detectable activity in the absence of an inhibitor. In some cases, a fusion polypeptide can include a HCoV-NL63 M^{pro} amino acid sequence that is at least 90% (e.g., at least 91%, at least 92%, at least 93%, at least 94%, at least 95%, at least 96%, at least 97%, at least 98%, or at least 99%, but not 100%) identical to the sequence set forth in residues 16 to 334 of SEQ ID NO:27, with the proviso that the HCoV-NL63 M^{pro} polypeptide has detectable activity in the absence of an inhibitor.

In some cases, a fusion polypeptide can include a HIV-1 Tat amino acid sequence that is at least 90% (e.g., at least 91%, at least 93%, at least 94%, at least 95%, at least 97% or at least 98%, but not 100%) identical to the sequence set forth in residues 347 to 418 of SEQ ID NO:1, residues 347 to 418 of SEQ ID NO:23, residues 343 to 414 of SEQ ID NO:25, or residues 344 to 415 of SEQ ID NO:27, with the proviso that the HIV-1 Tat polypeptide has transcriptional activator activity.

In some cases, a fusion polypeptide can include an eGFP amino acid sequence that is at least 90% (e.g., (e.g., at least 91%, at least 92%, at least 93%, at least 94%, at least 95%, at least 96%, at least 97%, at least 98%, or at least 99%, but not 100%) identical to the sequence set forth in residues 425 to 663 of SEQ ID NO:1, with the proviso that the eGFP polypeptide fluoresces when expressed separate from the fusion polypeptide. In some cases, a fusion polypeptide can include a luciferase amino acid sequence that is at least 90% (e.g., (e.g., at least 91%, at least 92%, at least 93%, at least 94%, at least 95%, at least 96%, at least 97%, at least 98%, or at least 99%, but not 100%) identical to the sequence set forth in residues 425 to 973 of SEQ ID NO:23, residues 421 to 969 of SEQ ID NO:25, or residues 422 to 970 of SEQ ID NO:27, with the proviso that the luciferase polypeptide luminesces when expressed separate from the fusion polypeptide.

This document also provides nucleic acid constructs encoding the modular reporter polypeptides described herein. The terms “nucleic acid” and “polynucleotide” are used interchangeably, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

An “isolated” nucleic acid molecule is a nucleic acid that is separated from other nucleic acids that are present in a genome, e.g., a plant genome, including nucleic acids that normally flank one or both sides of the nucleic acid in the genome. The term “isolated” with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as DNA that is incorporated into a vector, an autonomously replicating plasmid, a virus (e.g., a pararetrovirus, a retrovirus, lentivirus, adenovirus, or herpes virus), or the genomic DNA of a prokaryote or eukaryote. In addition, an isolated nucleic acid can include a recombinant nucleic acid such as a DNA molecule that is (or is part of) a hybrid or fusion nucleic acid (e.g., a nucleic acid encoding a fusion protein as described herein). A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made by any appropriate method, including, for example, chemical synthesis, polymerase chain reaction (PCR) and variations thereof (e.g., overlap extension PCR), or restriction cloning techniques. PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

An example of a nucleotide sequence encoding the representative fusion polypeptide having SEQ ID NO:1 is set forth in SEQ ID NO:2 (FIG. 1B). An example of a nucleotide sequence encoding the representative fusion polypeptide having SEQ ID NO:23 is set forth in SEQ ID NO:24 (FIG. 2B). An example of a nucleotide sequence encoding the representative fusion polypeptide having SEQ ID NO:25 is set forth in SEQ ID NO:26 (FIG. 3B). An example of a nucleotide sequence encoding the representative fusion polypeptide having SEQ ID NO:27 is set forth in SEQ ID NO:28 (FIG. 4B). In some cases, a nucleotide sequence encoding a fusion polypeptide provided herein can be at least 50% (e.g., at least 55%, at least 60%, at least 65%, at least 70%, at least 75%, at least 80%, at least 85%, at least 90%, at least 95%, at least 96%, at least 97%, at least 98%, or at least 99%) identical to the sequence set forth in SEQ ID NO:2, SEQ ID NO:24, SEQ ID NO:26, or SEQ ID NO:28. In some cases, a nucleotide sequence (e.g., a viral nucleotide sequence) can be codon optimized for expression in mammalian cells. It is to be noted that codon optimization of a wild type sequence can result in an optimized nucleotide sequence with about 50% to about 90% (e.g., about 50% to about 70%, about 60% to about 80%, or about 70% to about 90%) sequence identity to the wild type sequence, while the amino acid sequence(s) encoded by the optimized nucleotide sequence can have at least 90% sequence identity to the wild type amino acid sequence(s).

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq c:\seq1.txt -j c:\seq2.txt -p blastn -o c:\output.txt -q −1 -r 2. To compare two amino acid sequences, the options of Bl2seq are set as follows: -i is set to a file containing the first amino acid sequence to be compared (e.g., C:\seql.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq c:\seql.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:2), or by an articulated length (e.g., 100 consecutive nucleotides or amino acid residues from a sequence set forth in an identified sequence), followed by multiplying the resulting value by 100. For example, a nucleotide sequence that has 2000 matches when aligned with the sequence set forth in SEQ ID NO:2 is 99.4 percent identical to the sequence set forth in SEQ ID NO:2 (i.e., 2000/2013×100=99.4). It is noted that the percent sequence identity value is rounded to the nearest tenth. For example, 75.11, 75.12, 75.13, and 75.14 are rounded down to 75.1, while 75.15, 75.16, 7.17, 75.18, and 7.19 are rounded up to 7.2. It also is noted that the length value will always be an integer.

Recombinant nucleic acid constructs (e.g., vectors) also are provided herein. A “vector” is a replicon, such as a plasmid, phage, or cosmid, into which another DNA segment (e.g., a sequence encoding a fusion polypeptide) may be inserted so as to bring about the replication of the inserted segment. Generally, a vector is capable of replication when associated with the proper control elements. Suitable vector backbones include, for example, plasmids, viruses, artificial chromosomes, BACs, YACs, or PACs. The term “vector” includes cloning and expression vectors, as well as viral vectors and integrating vectors. An “expression vector” is a vector that includes one or more expression control sequences, and an “expression control sequence” is a DNA sequence that controls and regulates the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, WI), Takara Bio USA (Mountain View, CA), Stratagene (La Jolla, CA), Invitrogen/Life Technologies (Carlsbad, CA), ThermoFisher Scientific (Waltham, MA), and New England Biolabs (Ipswich, MA).

The terms “regulatory region,” “control element,” and “expression control sequence” refer to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, and other regulatory regions that can reside within coding sequences, such as secretory signals, Nuclear Localization Sequences (NLS) and protease cleavage sites. “Operably linked” means incorporated into a genetic construct so that expression control sequences effectively control expression of a coding sequence of interest. A coding sequence is “operably linked” and “under the control” of expression control sequences in a cell when RNA polymerase is able to transcribe the coding sequence into RNA, which if an mRNA, then can be translated into the protein encoded by the coding sequence. Thus, a regulatory region can modulate, e.g., regulate, facilitate or drive, transcription in the plant cell, plant, or plant tissue in which it is desired to express a modified target nucleic acid.

A promoter is an expression control sequence composed of a region of a DNA molecule, typically within 1000 nucleotides upstream of the point at which transcription starts (generally near the initiation site for RNA polymerase II). Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element. Any suitable promoter can be used to drive expression of the fusion polypeptides provided herein. For example, the promoter can be a constitutive promoter [e.g., a cytomegalovirus (CMV) promoter], or an inducible promoter.

In some cases, this document provides cells containing the nucleic acid constructs described herein. For example, a population of cells can be stably or transiently transfected with a nucleic acid encoding a fusion reporter polypeptide provided herein. In some cases, the cells can be cultured under conditions appropriate to allow expression of the reporter encoded by the nucleic acid. Any appropriate cells can be transfected with a nucleic acid construct provided herein (e.g., primary cells, or cell lines such as HEK-293 cells, HeLa cells, or CHO cells). In some cases, lentiviral transduction can be used to achieve stable expression of a nucleic acid construct provided herein.

This document also provides kits containing the nucleic acid constructs described herein, or containing cells transfected with the nucleic acid constructs described herein. The nucleic acid or the cells can be packaged in any appropriate media and maintained under any appropriate conditions for storage and shipping. For example, a nucleic acid construct can be dissolved in a buffer (e.g., Tris buffer or TE buffer, which contains Tris-HCl and EDTA) and frozen. Cells also can be frozen in an appropriate medium, typically with a cryoprotective agent such as DMSO or glycerol.

In some cases, this document provides methods for using the polypeptides, nucleic acids, and cells described herein. For example, this document provides methods for assessing the ability of agents to inhibit activity of the protease within a modular reporter polypeptide provided herein. In some cases, the methods provided herein also can be used to characterizing the relative strength of a protease inhibitor.

For example, a method provided herein can include providing a cell that has been transfected with, and expresses a nucleic acid construct encoding a modular reporter polypeptide as described herein. In some cases, the method also can include transfecting the cell with the nucleic acid construct. The level of reporter activity in the cell can be determined (e.g., by visualization or quantification) and compared to a control level of reporter activity. If the level of reporter activity in the test cell is increased as compared to the level of reporter activity in the control cell (e.g., determined by visualization or quantification), the agent can be identified as being an inhibitor of the protease. If the level of reporter activity in the test cell is not increased as compared to the control level of reporter activity, then the agent may not be identified as an inhibitor of the protease.

Any appropriate control can be used for the methods provided herein. In some cases, for example, a control level of reporter activity can be the level of reporter activity observed or measured in the cell prior to contacting the cell with the candidate inhibitor. In some cases, the control level of reporter activity can be the level of reporter activity observed or measured in a corresponding cell that was transfected with and expresses the nucleic acid construct, but was not contacted with the agent.

Any suitable agent can be tested as a potential protease inhibitor. In some cases, for example, the agent can be a small molecule (e.g., GC376, boceprevir, or similar compounds, or a compound such as ebselen or carmofur). Other small organic molecules (e.g., drugs or drug-like compounds), nucleic acids, nucleic-acid-based aptamers, peptide, peptide-mimetics, antibodies, or antigen-binding fragments (e.g., intrabodies) also can be used.

In some cases, for example, an agent can be an anti-protease antibody or an antigen-binding fragment thereof. The term “antibody” as used herein encompasses include intact molecules (e.g., polyclonal antibodies, monoclonal antibodies, humanized antibodies, or chimeric antibodies) as well as fragments thereof (e.g., single chain Fv antibody fragments, Fab fragments, and F(ab)₂ fragments) that are capable of binding to an epitopic determinant of a protease. An epitope is an antigenic determinant on an antigen to which the paratope of an antibody binds. Epitopic determinants typically consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). Polyclonal antibodies are heterogeneous populations of antibody molecules that are contained in the sera of the immunized animals. Monoclonal antibodies are homogeneous populations of antibodies to a particular epitope of an antigen.

Antibodies having specific binding affinity for a protease (e.g., M^{pro}) can be produced using, for example, standard methods. See, for example, Dong et al., Nature Med 8:793-800, 2002. In general, a protease polypeptide can be recombinantly produced or can be purified from a biological sample, and then can be used to immunize an animal in order to induce antibody production. Antibody fragments can be generated by any suitable technique. For example, F(ab′)₂ fragments can be produced by pepsin digestion of an antibody molecule, and Fab fragments can be generated by reducing the disulfide bridges of F(ab′)₂ fragments. Alternatively, Fab expression libraries can be constructed. See, for example, Huse et al., Science 246:1275, 1989. Once produced, antibodies or fragments thereof can be tested for recognition of a target protease by standard immunoassay methods, including ELISA techniques, radioimmunoassays, and western/immuno blotting.

In some cases, this document provides methods for identifying a protease as containing a mutation that reduces or eliminates activity of the protease. For example, a method can include providing a cell transfected with a nucleic acid that encodes a modular reporter polypeptide provided herein, where the amino acid sequence of the protease polypeptide within the modular reporter has one or more (e.g., one, two, three, four, five, or more than five) mutations with respect to the amino acid sequence of the wild type protease. In some cases, the method also can include transfecting the cell with the nucleic acid. The level of reporter activity in the cell can be determined and compared to the level of reporter activity in a control cell expressing a corresponding reporter polypeptide that includes a protease sequence without the mutation(s). If the level of reporter activity in the test cell is increased as compared to the level of reporter activity in the control cell, the mutation(s) in the protease can be identified as inhibitors of protease activity. If the level of reporter activity in the test cell is not increased as compared to the level of reporter activity in the control cell, the mutation(s) in the protease may not be identified as inhibitors of protease activity.

An “increase” in activity of a modular reporter polypeptide provided herein can be any increase in the level of reporter activity detected (e.g., by visualization or quantification), as compared to the level of reporter activity detected in the absence of the inhibitory agent or the mutation being assessed. In some cases, for example, an “increased” level of reporter activity can be an increase of at least 10% (e.g., at least 20%, at least 30%, at least 50%, or at least 100%) in the level of reporter activity in a test cell as compared to a control cell that was not treated with an inhibitor or that contains a reporter polypeptide in which the protease portion does not contain a mutation.

The invention will be further described in the following examples, which do not limit the scope of the invention described in the claims.

### EXAMPLES

### Example 1—Materials and Methods

Plasmid construction: To generate the Src-M^{pro}-Tat-eGFP construct, the M^{pro} (Nsp5), Tat, and eGFP coding sequences were amplified from existing vectors and fused using overlap extension PCR. The final reaction added the 5′-myristolation sequence from Src and HindIII and NotI sites for restriction and ligation into similarly digested pcDNA5/TO (Thermo Fisher Scientific, #V103320). Wild type and catalytic mutant Nsp5 were amplified from pLVX-EF1alpha-nCoV2019-nsp5-2xStrep-IRES-Puro (Gordon et al., Nature 583:459-468, 2020) using 5′-GTGGGTCATCTATCACCTCAGCTGTTTTGCAGTCTGGTTTTAGGAAAATGGCGTTCC-3′ (SEQ ID NO:3) and 5′-CCCCCTGACCCGGTACCCTTGATTGTTCTTTTCACTGCACTCTGGAAAGTGACCCCACTG-3′ (SEQ ID NO:4). The Nsp5 cleavage site double mutant was amplified from the same template using 5′-GTGGGTCATCTATCACCTCAGCTGTTTTGGCTTCTGGTTTTAGGAAAATGGCGTTCC-3′ (SEQ ID NO:5) and 5′-CCCCCTGACCCGGTACCCTTGATTGTTCTTTTCACTGCACTCGCGAAAGTGACCCCACTG-3′ (SEQ ID NO:6). The sequence encoding HIV-1 Tat residues 1-72 was amplified from a HIV-1 BH10 full molecular clone (Sarver et al., Science 247:1222-1225, 1990) using 5′-AGAACAATCAAGGGTACCGGGTCAGGGGGCAGCGGAGGGATGGAGCCAGTAGATCCTAGA-3′ (SEQ ID NO:7) and 5′-GGTGGCGATGGATCCCGGCTGCTTTGATAGAGAAACTTGATGAGTCT-3′ (SEQ ID NO:8). The eGFP coding sequence was amplified from pcDNA5/TO-A3B-eGFP (Burns et al., Nature 494:366-370, 2013) using 5′-AGACTCATCAAGTTTCTCTATCAAAGCAGCCGGGATCCATCGCCACC-3′ (SEQ ID NO:9) and 5′-GACTCGAGCGGCCGCTTTACTTGTACAGCTCGTCCAT-3′ (SEQ ID NO:10). The Src myristoylation sequence (Song et al., Cell Mol Biol (Noisy-le-grand) 43:293-303, 1997) was added using 5′-AAGCTTGCCACCATGGGCAGCAGTAAGAGTAAACCGAAAGATGGAGGCGGTGGGTCATCTATCACCTCAGCT-3′ (SEQ ID NO:11) and the eGFP reverse primer. Sanger sequencing confirmed the integrity of all constructs.

Cell culture and flow cytometry: 293T cells were maintained at 37° C./5% CO₂ in RPMI-1640 (Gibco #11875093) supplemented with 10% fetal bovine serum (Gibco #10091148) and penicillin/streptomycin (Gibco #15140122). 293T cells were seeded in a 24-well plate at 1.5×10⁵ cells/well and transfected 24 hours later with 200 ng of the wild type or mutant chimeric reporter construct (TranslT-LT1, Minis #MIR2304). 48 hours post-transfection, cells were washed twice with PBS and resuspended in 500 μL PBS. One-fifth of the cell suspension was transferred to a 96-well plate, mixed with TO-PRO3 ReadyFlow Reagent for live/dead staining per the manufacturer's protocol (Thermo Fisher Scientific #R37170), incubated at 37° C. for 20 minutes, and analyzed by flow cytometry (BD LSRFortessa). The remaining four-fifths of the cell suspension was pelleted, resuspended in 50 μL PBS, mixed with 2× reducing sample buffer, and analyzed by immunoblotting.

Fluorescent Microscopy: 50,000 293T cells were plated in a 24 well plate and allowed to adhere overnight. The next day, cells were transfected with 150 ng of each plasmid and 50 ng of an NLS-mCherry vector as a transfection and imaging control. Images were collected 48 hours post-transfection at 10× magnification using an EVOS FL Color Microscope (Thermo Fisher Scientific).

Immunoblots: Whole cell lysates in 2× reducing sample buffer (125 mM Tris-HCl pH 6.8, 20% glycerol, 7.5% SDS, 5% 2-mercaptoethanol, 250 mM DTT, and 0.05% bromophenol blue) were denatured at 98° C. for 15 minutes, fractionated using SDS-PAGE (4-20% Mini-PROTEAN gel, Bio-Rad #4568093), and transferred to a polyvinylidene difluoride (PVDF) membrane (Millipore #IPVH00010). Immunoblots were probed with mouse anti-GFP (1:10,000 JL-8, Clontech #632380) and rabbit anti-β-actin (1:10,000 Cell Signaling #4967) followed by goat/sheep anti-mouse IgG IRDye 680 (1:10,000 LI-COR #926-68070) or goat anti-rabbit IgG-HRP (1:10,000 Jackson Labs #111-035-144). HRP secondary antibody was visualized using the SuperSignal West Femto Maximum Sensitivity Substrate (Thermo Fisher #PI34095). Images were acquired using the LI-COR Odyssey Fc imaging system.

### Example 2—Gain-of-Function Assay for M^{pro} Inhibition in Living Cells

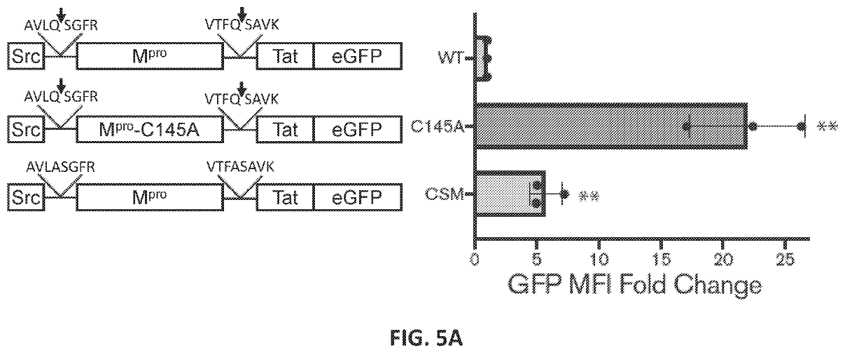

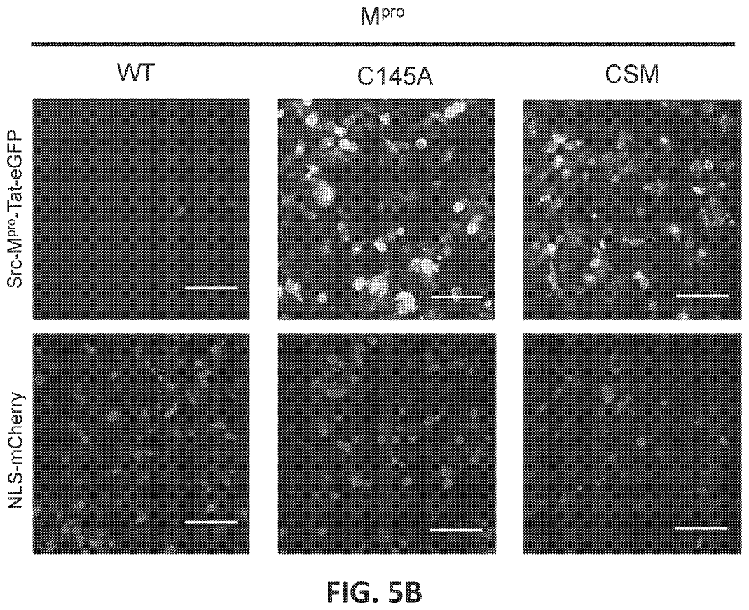

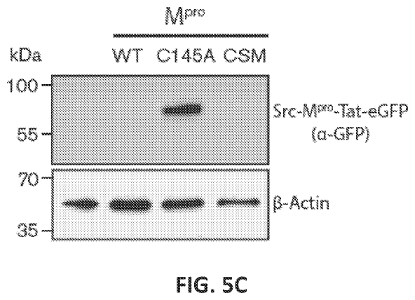

Studies were carried out in an attempt to create a chromosomal reporter for SARS-CoV-2 infectivity, analogous to HIV-1 single cycle assays. During this work, an apparently non-functional chimeric protein was constructed that consisted of an N-terminal myristoylation domain from Src kinase, the full M^{pro} amino acid sequence with cognate N- and C-terminal self-cleavage sites, the HIV-1 transactivator of transcription (Tat), and eGFP (FIG. 5A). Transfection into 293T cells failed to yield green fluorescence by flow cytometry or microscopy (FIGS. 5A and 5B). Surprisingly, however, an otherwise identical construct with a catalytic site mutation in M^{pro} (C145A) resulted in high levels of fluorescence, suggesting that auto-proteolytic activity was required for the apparent lack of expression of the wild type construct. This possibility was further supported by fluorescence of a cleavage site double mutant construct (CSM), in which the conserved glutamines required for M^{pro} auto-proteolysis were changed to alanines (corresponding to Nsp4-Q500A and M^{pro}/Nsp5-Q306A). The double mutant showed less fluorescence than the M^{pro} C145A catalytic mutant, potentially due to recognition of alternative cleavage sites. This interpretation was underscored by immunoblots showing strong expression of the full chimeric M^{pro} C145A catalytic mutant protein but no visible expression of the wild type construct (FIG. 5C). Although the CSM yielded fluorescence, the full-length chimeric protein was undetectable by anti-eGFP immunoblotting (FIGS. 5A-5C).

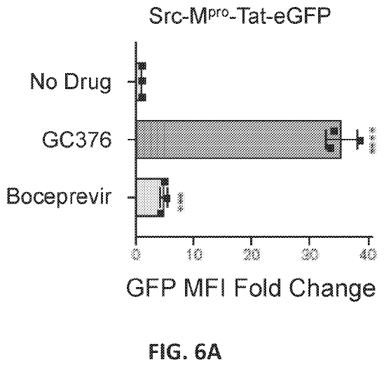

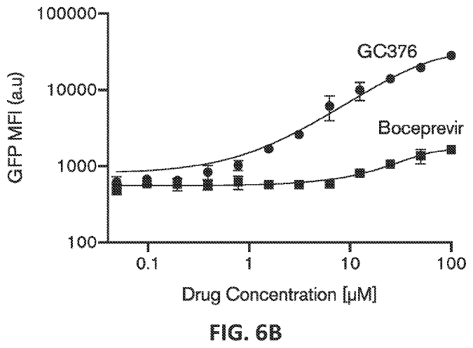

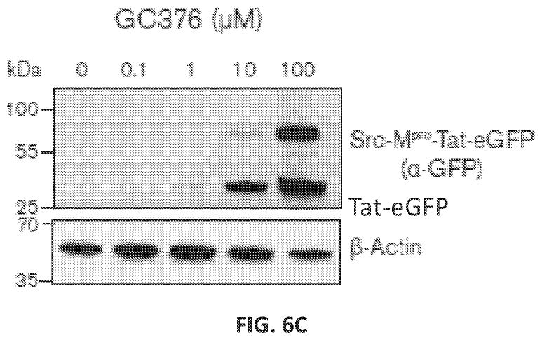

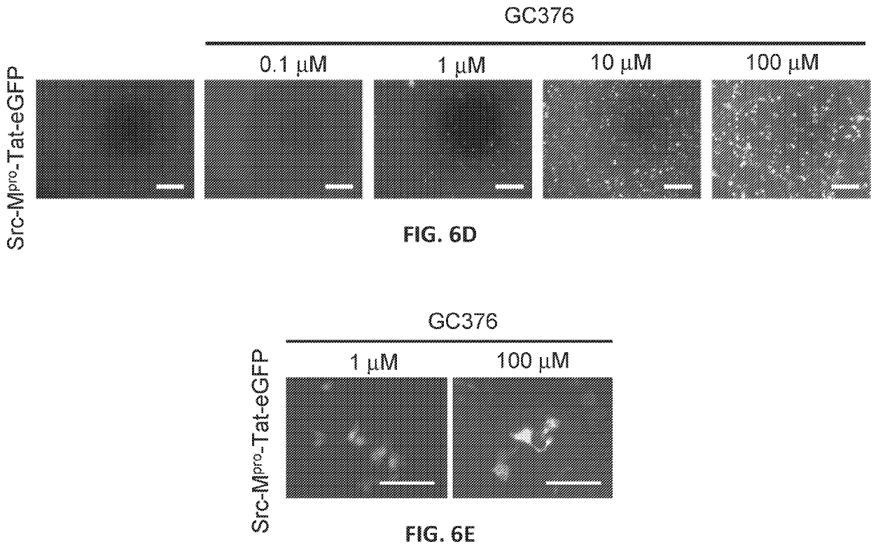

Multiple small molecule inhibitors of M^{pro} have been described, including GC376 and boceprevir (Gioia et al., Biochem Pharmacol 182:114225, 2020). GC376 was developed against a panel of 3C and 3C-like cysteine proteases, including feline coronavirus M^{pro} (Kim et al., J Virol 86:11754-11762, 2012; and Pedersen et al., J Feline Med Surg 20:378-392, 2018). Boceprevir was developed as an inhibitor of the NS3 protease of hepatitis C virus (Hazuda et al., supra; Venkatraman et al., J Med Chem 49:6074-6086, 2006; and Lamarre et al., Nature 426:186-189, 2003). These small molecules also have also been co-crystalized with SARS-CoV-2 M^{pro}, and their binding sites have been defined (Fu et al., supra; and Ma et al., Cell Res 30:678-692, 2020). Thus, studies were conducted to determine whether a high dosage of these compounds could mimic the genetic mutants described above and restore fluorescence activity of the wild type construct. Interestingly, 50 μM GC376 caused a strong restoration of expression and fluorescence of the wild type construct (FIG. 6A). In comparison, 50 μM boceprevir caused a weaker but still significant effect. The potencies of GC376 and boceprevir were confirmed in dose response experiments, with both fluorescent microscopy and immunoblotting as experimental readouts (FIGS. 6B and 6C). These studies demonstrated that the assay successfully distinguishes the potencies of different protease inhibitors. Interestingly, at high concentrations of GC376 (100 μM), the subcellular localization of the wild type chimeric protein phenocopied the C145A catalytic mutant, with predominantly cytoplasmic membrane localization due to the N-terminal myristoyl anchor (FIGS. 6D and 6E). At lower concentrations (1 μM), however, the eGFP signal was mainly nuclear—consistent with partial M^{pro} activity and import of the Tat-eGFP portion of the chimera into the nuclear compartment through the NLS of Tat (FIGS. 6D and 6E) (Efthymiadis et al., J Blot Chem 273:1623-1628, 1998). These subcellular localization data were reflected by immunoblots in which a Tat-eGFP band predominated at low drug concentrations, while full-length Src-M^{pro}-Tat-eGFP was clearly visible at high concentrations (FIG. 6C).

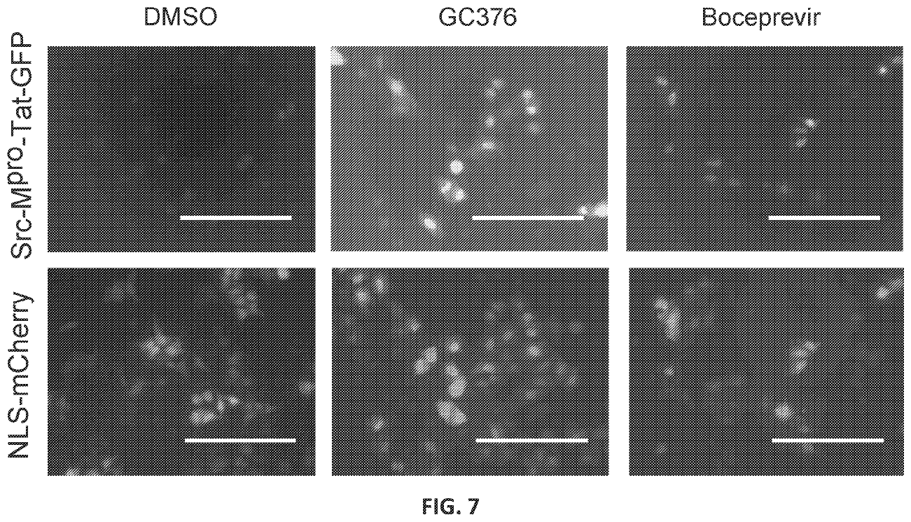

The Src-M^{pro}-Tat-eGFP construct provides a quantitative (“Off-to-On”) fluorescent read-out of genetic and pharmacologic inhibitors of SARS-CoV-2 M^{pro} activity. The system is modular and is likely to be equally effective with sequences derived from other N-myristoylated proteins, such as the ARF GTPases and HIV-1 Gag, with sequences from other proteases (e.g., closely related coronavirus proteases such as MERS and SARS M^{pro}or more distantly related viral proteases such as HCV NS3/4a and picornavirus 3C), and with the full color spectrum of fluorescent proteins or luminescent proteins. The system also is cell-autonomous, as similar results were obtained using both 293T and HeLa cell lines (FIG. 7).

The molecular explanation for the instability of the wild type chimeric construct is not clear. Without being bound by a particular mechanism, however, the instability might be due to protease-dependent exposure of an otherwise protected protein degradation motif (degron). Regardless of the full mechanism, the gain-of-function system described herein for protease inhibitor characterization and development in living cells is likely to have immediate and broad utility in academic and pharmaceutical research.

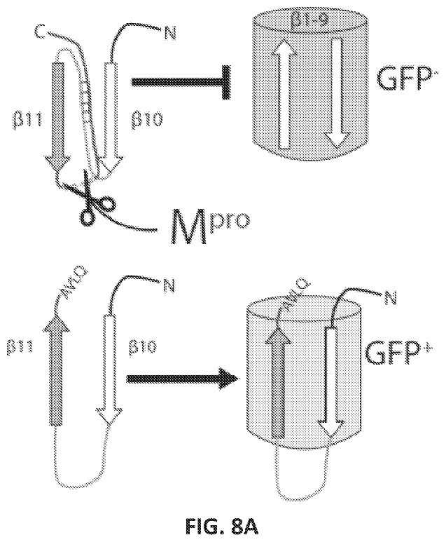

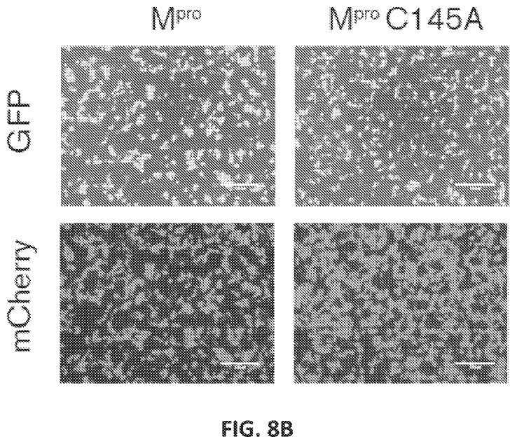

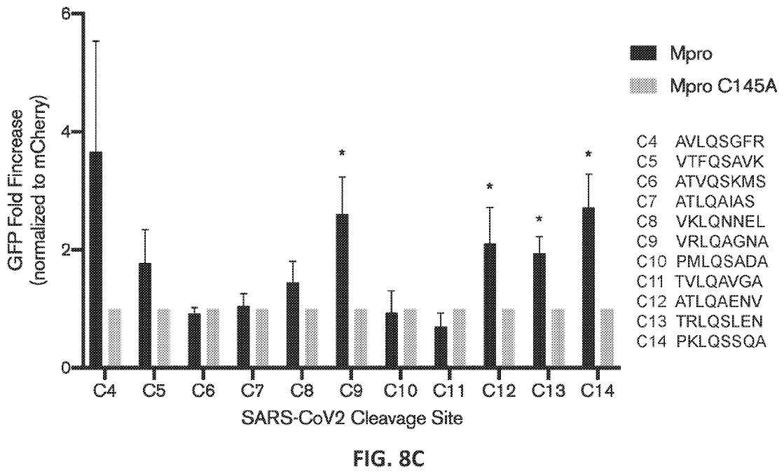

Existing assays for SARS-CoV-2 M^{pro} activity in living cells are non-specific and/or less sensitive. One assay is a simple measure of cell death with M^{pro} overexpression resulting in toxicity (Resnick et al., doi org/10 1101/2020.08.29 272804, 2020). The application of this assay for high throughput screening is limited due to incomplete cell death (resulting in low signal/noise) and issues dissociating pro inhibition from small molecule modulators of cell death pathways including apoptosis. A different assay (“FlipGFP”) uses M^{pro} activity to “flip-on” GFP fluorescence (Froggatt et al., J Virol 94(22):e01265-20, 2020; illustrated in FIG. 8A). Although this assay provides some specificity for pro catalytic activity, it shows a narrow dynamic range for GC376, making it poorly equipped for inhibitor optimization or high-throughput screening to identify additional inhibitors.

The FlipGFP system yielded substantial levels of background in the absence of pro activity (i.e., the pro signal was only 2-fold higher than background noise; FIGS. 8B and 8C). However, the most important distinction between any live cell pro inhibitor assay described elsewhere (e.g., FlipGFP) and the system described herein is the readout for chemical inhibition. The former assays measure signal diminution (which quickly run into background), while the assay provided herein provides a gain-of-function fluorescent signal that is far above negligible background levels. By reading-out an increase in eGFP signal that directly reflects the potency of M^{pro} inhibition, the present system provides stringent specificity for small molecules that target M^{pro} catalytic activity. Moreover, the assay provided herein helps to identify compounds that are cell permeable and non-toxic, as less permeable and toxic compounds are likely to yield less fluorescent signal and effectively drop from consideration. The assay provided herein therefore is an important contribution to the development of potent drugs to combat the current SARS-CoV-2 pandemic, as well as future coronavirus zoonoses.

### Example 3 — Sensitivity of a Luciferase-Based Reporter vs. an eGFP Reporter

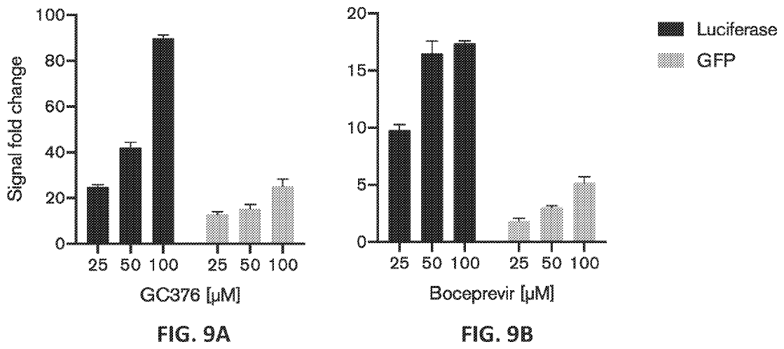

A Src-SARS2-M^{pro}-Tat-fLuc reporter (SEQ ID NO:23) containing a firefly luciferase domain was constructed, and its sensitivity was compared to that of the Src-SARS2-M^{pro}-Tat-eGFP reporter. [please fill in type of] cells were transfected with a construct encoding the eGFP-based reporter or the luciferase-based reporter, and treated with GC376 or boceprevir. As shown in FIGS. 9A and 9B, the luciferase-based reporter yielded higher relative levels of signal/activity in response to both GC376 (FIG. 9A) and boceprevir (FIG. 9B).

### Example 4—Function of Different Coronavirus M^{pro} Enzymes in the Reporter System

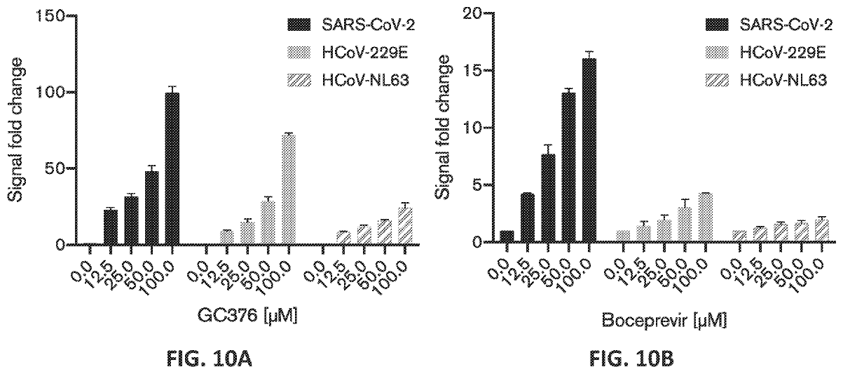

Reporter constructs containing several different coronavirus M^{pro} enzymes were generated and tested. Specifically, constructs encoding reporters containing SARS-CoV-2 M^{pro}, HCoV-229E M^{pro}, or HCoV-NL63 M^{pro} (reporter amino acid sequences set forth in SEQ ID NOS:23, 25, and 27, respectively) were generated and transfected into [please fill in type of] cells. The cells were treated with increasing concentrations of GC376 (FIG. 10A) or boceprevir (FIG. 10B). These studies demonstrated that the reporter containing SARS-CoV-2 M^{pro} yielded higher relative levels of signal/activity in response to both GC376 and boceprevir, followed by HCoV-229E M^{pro} and then HCoV-NL63 M^{pro}.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nucleic acid construct encoding a modular reporter polypeptide, wherein the modular reporter polypeptide comprises, in order from N-terminus to C-terminus:
a myristoylation motif,
a protease polypeptide,
a transactivator of transcription (Tat) sequence, and
a reporter polypeptide.

2. The nucleic acid of claim 1, wherein the myristoylation motif is a Src myristoylation motif, an ADP-ribosylation factor (ARF) GTPase myristoylation motif, a human immunodeficiency virus-1 (HIV-1) Gag myristoylation motif, or a myristoylated alanine-rich C kinase substrate (MARCKS) myristoylation motif.

3. (canceled)

4. The nucleic acid construct of claim 1, wherein the protease polypeptide is a SARS-CoV-2 Mpro polypeptide, a MERS Mpro polypeptide, a SARS Mpro polypeptide, a hepatitis C virus (HCV) NS3/4a protease polypeptide, a picornavirus 3C protease polypeptide, a HCoV-229E Mpro polypeptide, or a HCoV-NL63 Mpro polypeptide.

5. (canceled)

6. The nucleic acid construct of claim 1, wherein the Tat sequence comprises amino acids 1 to 72 of HIV-1 Tat.

7. The nucleic acid construct of claim 1, wherein the reporter is a fluorescent polypeptide or a luminescent polypeptide.

8-9. (canceled)

10. The nucleic acid construct of claim 1, wherein the modular reporter polypeptide further comprises a first linker sequence between the myristoylation motif and the protease polypeptide, a second linker sequence between the protease polypeptide and the Tat sequence, and a third linker sequence between the Tat sequence and the fluorescent reporter polypeptide.

11-14. (canceled)

15. A method for identifying an agent as being a protease inhibitor, wherein the method comprises:
providing a cell transfected with and expressing a nucleic acid construct encoding a modular reporter polypeptide, wherein the modular reporter polypeptide comprises, in order from N-terminus to C-terminus:
a myristoylation motif,
a protease polypeptide,
a Tat sequence, and
a reporter polypeptide;
contacting the cell with the agent;
determining a level of reporter activity in the cell;
comparing the level of reporter activity in the cell to a control level of reporter activity; and
identifying the agent as being an inhibitor of the protease when the level of reporter activity in the cell is higher than the control level of reporter activity.

16. The method of claim 15, wherein the reporter activity is fluorescence or luminescence.

17. The method of claim 15, wherein the control level of reporter activity is a level of reporter activity in the cell determined prior to the contacting step, or wherein the control level of reporter activity is a level of reporter activity in a corresponding cell transfected with and expressing the nucleic acid construct but not contacted with the agent.

18. (canceled)

19. The method of claim 15, wherein the myristoylation motif is a Src myristoylation motif, an ARF GTPase myristoylation motif, a HIV-1 Gag myristoylation motif, or a MARCKS myristoylation motif.

20. (canceled)

21. The method of claim 15, wherein the protease polypeptide is a SARS-CoV-2 Mpro polypeptide, a MERS Mpro polypeptide, a SARS Mpro polypeptide, a HCV NS3/4a protease polypeptide, a picornavirus 3C protease polypeptide, a HCoV-229E Mpro polypeptide, or a HCoV-NL63 Mpro polypeptide.

22. (canceled)

23. The method of claim 14, wherein the Tat sequence comprises amino acids 1 to 72 of HIV-1 Tat.

24-26. (canceled)

27. The method of claim 15, wherein the modular reporter polypeptide further comprises a first linker sequence between the myristoylation motif and the protease polypeptide, a second linker sequence between the protease polypeptide and the Tat sequence, and a third linker sequence between the Tat sequence and the fluorescent reporter polypeptide.

28-31. (canceled)

32. The method of claim 15, wherein the agent is a small molecule or an anti-Mpro antibody.

33. A method for identifying a protease as having a mutation that reduces activity of the protease, wherein the method comprises:
providing a cell transfected with and expressing a nucleic acid construct encoding a modular reporter polypeptide, wherein the modular reporter polypeptide comprises, in order from N-terminus to C-terminus:
a myristoylation motif,
a protease polypeptide, wherein the amino acid sequence of the protease polypeptide comprises a mutation with respect to a corresponding wild type amino acid sequence,
a Tat sequence, and
a reporter polypeptide;
determining a level of reporter activity in the cell;
comparing the level of reporter activity in the cell to a control level of reporter activity; and
identifying the agent as being an inhibitor of the protease when the level of reporter activity in the cell is higher than the control level of reporter activity.

34. The method of claim 33, wherein the reporter activity is fluorescence or luminescence.

35. The method of claim 33, wherein the control level of reporter activity is a level of reporter activity in a corresponding cell transfected with and expressing a nucleic acid construct that encodes a modular reporter polypeptide comprising a protease polypeptide having a wild type amino acid sequence.

36. The method of claim 33, wherein the myristoylation motif is a Src myristoylation motif, an ARF GTPase myristoylation motif, a HIV-1 Gag myristoylation motif, or a MARCKS myristoylation motif.

37. (canceled)

38. The method of claim 33, wherein the protease polypeptide is a SARS-CoV-2 Mpro polypeptide, a MERS Mpro polypeptide, a SARS Mpro polypeptide, a HCV NS3/4a protease polypeptide, a picornavirus 3C protease polypeptide, a HCoV-229E Mpro polypeptide, or a HCoV-NL63 Mpro polypeptide.

39. (canceled)

40. The method of claim 33, wherein the Tat sequence comprises amino acids 1 to 72 of HIV-1 Tat.

41-43. (canceled)

44. The method of claim 33, wherein the modular reporter polypeptide further comprises a first linker sequence between the myristoylation motif and the protease polypeptide, a second linker sequence between the protease polypeptide and the Tat sequence, and a third linker sequence between the Tat sequence and the fluorescent reporter polypeptide.

45-64. (canceled)
